(19) United States
(12) Patent Application Publication
(10) Pub. No.: US 2008/0074616 A1
(43) Pub. Date: Mar. 27, 2008

(54) Viewing a display of portable device close to eye

(76) Inventor: Suganda Jutamulia, Berkeley, CA (US)

Correspondence Address: SUGANDA JUTAMULIA, 2108 JEFFERSON AVENUE, BERKELEY, CA, 94703, US

(21) Appl. No.: 11/518,301

(22) Filed: Sep. 9, 2006

(51) Int. Cl. A61B 3/10

(52) U.S. Cl. 351/211

(57) ABSTRACT

For magnifying a display of a portable device, the display is placed at a distance less than the distance of the normal near point to the eye (about 25 cm). However, the image will be blurred since the eye cannot focus the image onto the retina. To form a magnified image onto the retina, a positive lens is added in front of the eye. With a positive lens in front of the eye, and the device is placed closer than the near point to the eye, a viewer can see clearly the magnified display. A 2.5″ (6.25 cm) display will be seen like a 32″ (80 cm) display or even larger.

### BACKGROUND

1. Field

This relates to viewing a small display, specifically to viewing a display of a portable electronic device placed close to the eye.

2. Description of Prior Art: Eyeglasses

Eyeglasses were invented several hundreds years ago. The human eye (FIG. 1(a)) comprises a cornea 20 and a crystalline lens 22 to form an image on the retina 24. For a normal person, when an object is located at infinity, the incident light from the object is comprises virtually parallel rays that are focused by the combination of cornea 20 and crystalline lens 22 onto retina 24 as shown. Since the image is focused on the retina, the object is clearly seen.

When the object is moved closer to the eye, the lines of light from the object will diverge so that, if the lens remains unchanged, it will focus the light behind the retina. To bring back the focus onto the retina, the ciliary eye muscles surrounding the lens contract so that lens 22 bulges to decrease its focal length. This moves the focus of the image back onto retina 24 (FIG. 1(b)).

However, there is a minimum focal length of lens 22 corresponding to the maximum muscle contraction, such that the focal length cannot be further decreased beyond it. In other words, there is a minimum distance between an object and the eye that the object can still be clearly seen. The closest point on which the eye can focus is known as the near point. The closest point of a normal person is about 25 cm from the eye. FIG. 1(b) shows a point object at near point A being focused onto retina 24 at C. The distance AB from the near point to cornea 20 is about 25 cm for a normal person. If the object is located less than 25 cm from the eye, the image cannot be formed onto retina 24, and the viewed object becomes blurred.

For a person with hyperopia (farsightedness) or presbyopia (aged eye), the near point is at a distance greater than 25 cm. For example, it might be 60 cm. An object at point D, seen by a person with hyperopia (FIG. 2(a)), where DB is 60 cm and B is the vertex of cornea 20, can be focused at C on retina 24. However, at a normal near point A, the point object is focused at E behind retina 24 as shown in FIG. 2(b). Therefore a hyperopic person cannot see clearly an object at near normal point A, where AB is 25 cm.

To enable a hyperopic person to see clearly an object at normal near point A, an eyeglass 26 (FIG. 2(c)), which is a positive lens, is added in front of the eye. Eyeglass 26, cornea 20, and crystalline lens 22 jointly focus a point object at normal near point A at C on retina 24 as shown in FIG. 2(c). Nevertheless, insofar as I am aware, no one has attempted to make eyeglasses that enable a person to see clearly an object closer than the normal near point, which is about 25 cm.

3. Description of Prior Art: Small Displays of Portable Electronic Devices

Many portable electronic imaging or video devices having relatively small-size liquid crystal displays (LCDs) have been available. These devices include cell phones, digital cameras, video game players, camcorders, pocket TVs, and most recently, portable video players such as those sold under the trademarks iPod by Apple Computer Inc. (www.store.apple.com) and Zen by Creative Technology Ltd. (www.creative.com). It is hard to view the display of these portable devices, especially to view video or film, which is produced and formatted for displaying on a large screen.

Accordingly, many devices and methods have been proposed to magnify the display of these portable devices. These include U.S. Pat. No. 4,443,819 (1984) to Funada et al. for a pocket TV; U.S. Pat. No. 5,075,799 (1991) to Pine et al. for a pager; U.S. Pat. No. 4,991,935 (1991) to Sakurai, and U.S. Pat. No. 5,119,239 (1992) and U.S. Pat. No. 5,307,209 (1994) to Iaquinto et al. for a video game player; U.S. Pat. No. 6,067,459 (2000) to Lincoln et al., and U.S. Pat. No. 6,222,686 (2001) to Richard, for a cell phone. The disclosed devices and methods share a common idea, which is to mount a magnifier (positive lens) in front of the display. The display with an added magnifier is then viewed at a normal viewing distance, i.e., a distance greater than the distance of the normal near point to the eye (25 cm).

Visor-type magnifiers are known, but these are used to magnify an object placed at the near point or farther from the eye (≧25 cm). Thus the visor-type magnifiers lens and the object are relatively far from the eye. In fact a visor usually can be worn over eyeglasses.

However, insofar as I am aware, no device or method has been taught that enables one with normal vision to view a display at a position closer than the normal near point to the eye.

### SUMMARY

In accordance with one embodiment, a display is viewed at a position closer than the normal near point to the eye and eyeglasses are provided to focus the otherwise blurred image.

### DRAWING FIGURES

FIG. 1(a) (prior art) shows the image formation of a normal person when a point object is at infinity, and FIG. 1(b) (prior art) shows the object at the normal near point.

FIG. 2(a) (prior art) shows the image formation of a hyperopic person when a point object is at the hyperopic near point, FIG. 2(b) (prior art) shows the object at the normal near point, both without wearing eyeglasses, and FIG. 2(c) (prior art) shows the object at the normal near point with the viewer wearing eyeglasses.

FIG. 3(a) shows a small screen and a large screen at the normal near point, and FIG. 3(b) shows a small screen located closer than the normal near point to the eye, in accordance with one embodiment.

FIG. 4(a) shows the image formation of a normal person when a display is placed closer than the normal near point without and FIG. 4(b) shows the image formation with an added positive lens, in accordance with one embodiment.

FIG. 5 shows a person with normal vision wearing a pair of reading eyeglasses viewing a display at a distance closer than the distance of the normal near point to the eye, in accordance with one embodiment.

### REFERENCE NUMERALS IN DRAWINGS

| | |
|---|---|
| 20 cornea | 22 crystalline lens |
| 24 retina | 26 eyeglass |
| 28 small screen | 30 large screen |
| 32 eye | 34 positive lens |
| 36 viewer | 38 reading eyeglasses |
| 40 portable device | |

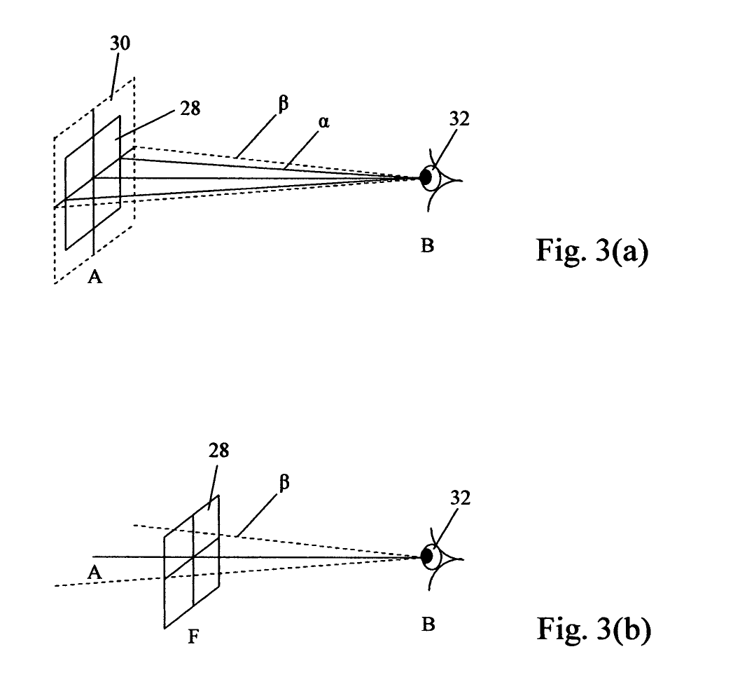

### FIG. 3—Description and Operation

FIG. 3(a) shows two screens 28 and 30 at normal near point A, which is 25 cm from an eye 32. The view angles of screens 28 and 30 are α and β, respectively. If small screen 28 is moved toward eye 32 so that view angle is β as shown in FIG. 3(b), small screen 28 at F will be seen as large as large screen 30 when it is at normal near point A Distance FB is less than 25 cm, where eye 32 is located at B.

To produce the view angle of small screen 28 β as shown in FIG. 3(b), distance FB must be AB×(tan α/2)/(tan β/2). To magnify small screen 28 two times, the distance to eye 32 must be decreased by half, so that FB is AB/2. To magnify screen 28 by k times (k is a magnification factor), the distance to eye 32 is divided by k, so that FB is AB/k.

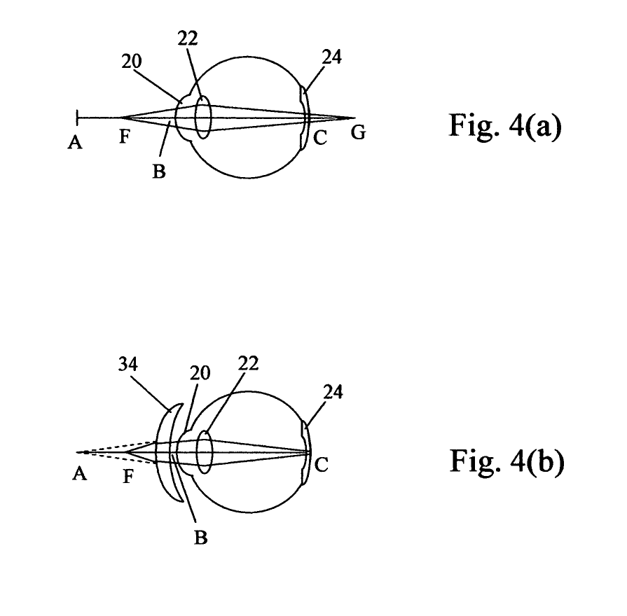

### FIG. 4—Description and Operation

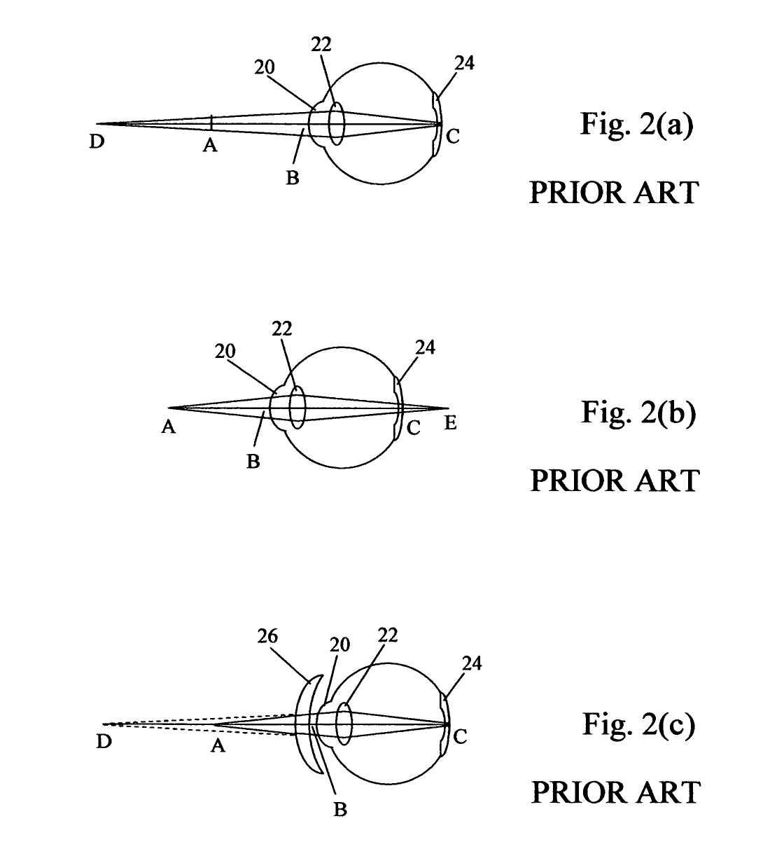

However, when an object is located at F (FIG. 4(a)), which is closer than normal near point A, the eye muscles cannot contract sufficiently to form an image that is focused on the retina. The image is formed at G, a point behind retina 24 as shown. To form an image on retina 24, a positive lens 34 (FIG. 4(b)), which is a magnifying glass, is added in front of the eye. The combination of positive lens 34, cornea 20, and crystalline lens 22 focuses the object at C on retina 24 as shown in FIG. 4(b). Positive lens 34 is similar to eyeglass 26 shown in FIG. 2(c). Positive lens 34 can also be mounted in an eyeglasses frame that can be worn by a viewer.

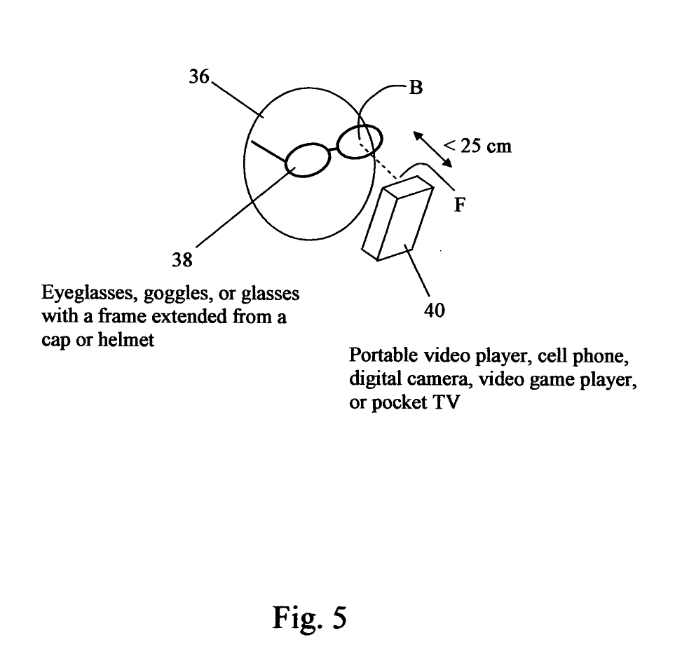

### FIG. 5—Description and Operation

FIG. 5 shows one embodiment employing the principle of FIG. 3. Since positive lens 34 of FIG. 4(b) is similar to eyeglass 26 of FIG. 2(c), a viewer 36 can wear a pair of reading eyeglasses 38 with sufficient dioptric power, for example +2.75 D (Diopter), to view a display of a portable device 40 at a distance FB less than 25 cm.

Reading eyeglasses 36 can also be replaced by goggles, or glasses with a frame extended from a cap or helmet. Portable device 40 can be a portable video player, a cell phone, a digital camera, a video game player, or a pocket TV.

For example, to magnify the display (2.5″ or 6.25 cm in diagonal) of an iPod video player, the video player is held at a distance less than the distance of the normal near point to the eye. If the normal near point is 25 cm from the eye, to magnify the display two times, the video player is held at 12.5 cm from the eye. To magnify the display three times, the video player is held at 8.33 cm from the eye. And so on.

The view angle of a screen in a theater is usually between 260 to 360, and the typical value is 30°. A 2.5″ (6.25 cm) diagonal display has 2″ (5 cm) width and 1.5″ (3.75 cm) height. To get 30° view angle, the display has to be placed at a distance of 9.25 cm from the eye (tan 15°=2.5 cm/9.25 cm). So the viewer will see the screen as if watching a movie in theater. However, since 9.25 cm is less than the distance of a normal near point, a normal person cannot see the display clearly as shown in FIG. 4(a). To see the display clearly, a positive lens, which is a magnifying glass, is added in front of the eye (FIG. 4(b)).

To focus the image of an object at 9.25 cm (point F in FIG. 4(b)) onto the retina of a person with normal vision, the added lens has to form a virtual image of that object at −25 cm (point A in FIG. 4(b)). A virtual image is an image that we see when looking through a magnifier or other refractive lens. The added lens forms a magnified image of the object at normal near point A, so that the eye will be able to see it clearly.

To accomplish this, the focal length of the positive lens must satisfy the lens equation given below.

$$\frac{1}{f} = \frac{1}{o} + \frac{1}{i} ,$$

where f is the focal length of lens, o is the object distance from the lens, and i is the image distance from the lens. Assume the lens is very close to the eye, object is +9.25 cm from the lens, and image is −25 cm from the lens. The focal length of lens will be 14.68 cm.

The dioptric power of a lens is defined as follows.

$$D [ Diopter ] = \frac{100}{f [ cm ]} ,$$

Where D is dioptric power in Diopter and f is focal length in cm. For 14.68 cm focal length, the dioptric power is +6.81 D.

The virtual image formed by the added lens becomes an object at 25 cm (point A in FIG. 4(b)) for the eye, which is the normal near point. With maximum contraction, the combination of cornea and crystalline lens in the eye will focus the image onto the retina properly. Thus the magnified display can be seen clearly by a person with normal vision, as large as a movie screen in theater.

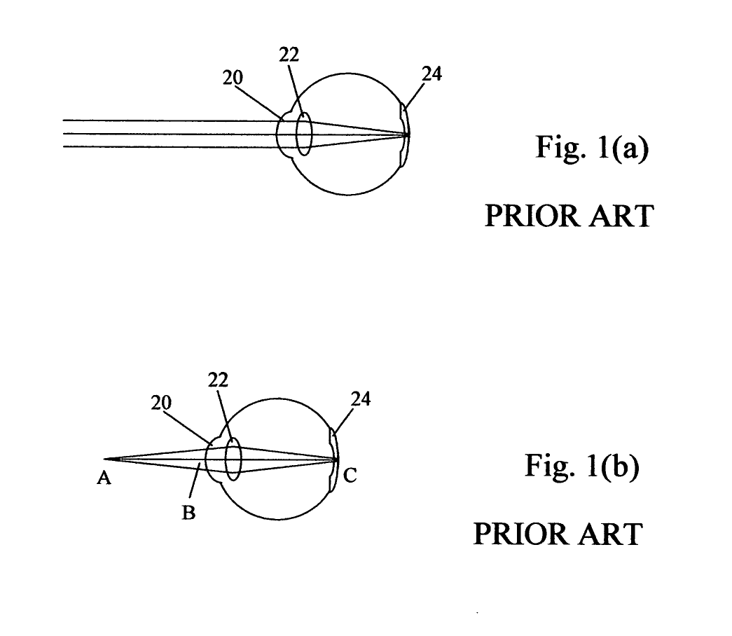

In one embodiment, the added positive lens forms a virtual image at infinity. This is done so that the virtual image formed by the added lens can be clearly seen by the eye without contraction (FIG. 1(a)). Referring to the same lens equation

$$\frac{1}{f} = \frac{1}{o} + \frac{1}{i} ,$$

for o=9.25 cm and i=−∞, the focal length will be 9.25 cm. The dioptric power of the positive lens is +10.81 D.

A display placed at 9.25 cm from the eye can be clearly seen by a viewer with normal vision using a positive lens placed in front of the eye. If the focal length of lens is 9.25 cm (+10.81 D), the magnified display can be seen without any eye contraction. If the focal length is 14.68 cm (+6.81 D), the magnified display still can be seen with maximum eye contraction. A viewer with normal vision needs some contraction to see clearly the magnified display using a lens with any focal length between 9.25 and 14.48 cm. Therefore, for a person with normal vision, a 30 view angle can be achieved for an iPod by placing the device at a distance 9.25 cm from the eye and using a magnifying lens having +6.81 D to +10.81 D power.

In one embodiment, the viewer wears a pair of reading eyeglasses, for example with +4 D power. Reading eyeglasses are sold over-the-counter in the dioptric power range of +1 D to +4 D. A prescription is required for eyeglasses over +4 D power. An eyeglass with +4 D power has a 25 cm focal length. Using the same lens equation again

$$\frac{1}{f} = \frac{1}{o} + \frac{1}{i} ,$$

where f=25 cm and i=−25 cm, one obtains o=12.5 cm.

By adding this eyeglass in front of the eye, the display can be moved up to a distance of only 12.5 cm from the eye such that the magnified display can still be clearly seen by a person with normal vision. At this position, the view angle is about 23°. This is equivalent to viewing a 32″ (80 cm) large-screen TV at a distance of about 2.05 m (6.83 feet). Thus the image will be as large as a large-screen TV at home, yet will be in focus.

### CONCLUSION, RAMIFICATIONS, AND SCOPE

Accordingly, the reader will see that the small display of a portable device can be magnified by placing the device closer than the normal near point to the eye and using a positive lens placed in front of the eye by a lens mounting means. The viewer can use a pair of non-prescription reading eyeglasses, which usually have the dioptric power range of +1 D to +4 D. The viewer can also use a pair of specially made eyeglasses, which have higher power as compared with over-the-counter reading glasses.

Portable devices with small displays include portable video players such as those sold under the trademarks iPod by Apple Computer Inc. and Zen by Creative Technology Ltd., cell phones, digital cameras, video game players, and pocket TVs.

Since the portable device is held at a distance closer than 25 cm, when the device is viewed in a plane, a train, a bus, or any public places, other people next to the viewer will not be able to see the display. Therefore it will protect the privacy of the viewer.

Although the description above contains many specificities, these should not be construed as limiting the scope of the invention but as merely providing illustrations of some of the presently preferred embodiments of this invention. Other preferred embodiments are possible, including:
lens 34 is part of a bifocal lens of eyeglasses;
only one lens 34 is mounted on an eyeglasses frame;
lens 34 at the glasses frame can be flipped up or down;
lens 34 is mounted on a goggles frame;
lens 34 is made in the form of contact lens;
lens 34 is mounted on a frame extended from a cap or helmet;
lens 34 is a tinted lens;
lens 34 is made from photochromic materials (materials that lighten indoors and darkens outdoors);
lens 34 can be a regular optical (smooth) lens or a Fresnel lens; and
the device can be monocular or binocular.

All embodiments mentioned above are for viewing a display of a portable device at a distance closer than the near point (<25 cm). In the mentioned embodiments, the magnifying lens is similar to an eyeglasses lens, i.e., it is placed very close to the eye.

Furthermore, one skilled in the art will be aware of a variety of means for mounting the positive lens or magnifying glass on a frame that holds the lens in front of the eye. Thus the scope of the invention should be determined by the appended claims and their legal equivalents, rather than by examples given.

1. A method for magnifying a display of a portable device, comprising:
(a) providing a positive lens for forming a magnified image onto the retina of an eye of a viewer;
(b) providing a mounting means for mounting said positive lens in front of the eye of said viewer;
(c) placing said display of said portable device closer than the near point to the eye of said viewer;
(d) mounting said positive lens in front of the eye of said viewer; and
(e) viewing said display of said portable device through said positive lens;
whereby said viewer can see a magnified image of said display of said portable device clearly even though it is closer than said near point to the eye of said viewer.

2. The method of claim 1 wherein said mounting means is a glasses frame.

3. The method of claim 1 wherein said mounting means is a goggles frame.

4. The method of claim 1 wherein said mounting means is extended from a cap or helmet.

5. The method of claim 1 wherein said positive lens is selected from the group consisting of a regular optical lens and a Fresnel lens.

6. The method of claim 1 where in said portable device is a portable video player.

7. The method of claim 1 where in said portable device is a cell phone.

8. The method of claim 1 where in said portable device is a digital camera.

9. The method of claim 1 where in said portable device is a video game player.

10. The method of claim 1 where in said portable device is a pocket TV.

11. A method using of reading eyeglasses having a positive dioptric value by a person with normal vision for magnifying a display of a portable device, comprising:
(a) wearing said reading eyeglasses having a positive dioptric value by said person with normal vision;
(b) placing said display of said portable device closer than the near point to the eye of said person with normal vision; and
(c) viewing said display of said portable device through said reading eyeglasses;
whereby said person with normal vision can see a magnified image of said display of said portable device clearly even though it is closer than said near point to the eye of said person with normal vision.

12. The method of claim 11 wherein said reading eyeglasses have dioptric power larger than +1 D.

13. The method of claim 11 wherein said device is placed closer than 25 cm in front of the eye of said person with normal vision.

14. The method of claim 11 where in said portable device is a portable video player.

15. The method of claim 11 where in said portable device is a cell phone.

16. The method of claim 11 where in said portable device is a digital camera.

17. The method of claim 11 where in said portable device is a video game player.

18. The method of claim 11 where in said portable device is a pocket television.
